(19) United States
(12) Patent Application Publication
(10) Pub. No.: US 2006/0234960 A1
(43) Pub. Date: Oct. 19, 2006

(54) Methods and compositions for increasing the safety and efficacy of albumin-binding drugs

(76) Inventors: Zhongmin Wang, Madison, AL (US); Joseph X. Ho, Madison, AL (US); Daniel C. Carter, Huntsville, AL (US)

Correspondence Address: STITES & HARBISON PLLC, 1199 NORTH FAIRFAX STREET, SUITE 900, ALEXANDRIA, VA, 22314, US

(21) Appl. No.: 11/365,805

(22) Filed: Mar. 2, 2006

Related U.S. Application Data
(60) Provisional application No. 60/657,427, filed on Mar. 2, 2005.

(51) Int. Cl. A61K 31/7048; A61K 31/704; A61K 31/549; A61K 31/43; A61K 31/4745; A61K 31/337; A61K 31/198; A61K 31/195; A61K 31/192

(52) U.S. Cl. 514/34; 514/192; 514/283; 514/305; 514/563; 514/569; 514/570; 514/567; 514/449; 514/223.5

(57) ABSTRACT

A method is provided for increasing the safety and efficacy of albumin-binding drugs such as those used as anti-cancer, anti-infective, or anti-hypertensive drugs, or for numerous other conditions. In the preferred method, the invention modulates those drugs which bind at the IB site on human serum albumin by co-administering a compound which is highly tolerable to humans and which can bind competitively with those albumin-binding drugs at the IB binding site so as to increase the safety and efficacy of the drug. The invention is advantageous in that by administering the highly tolerable compound in a sufficient amount to compete with the targeted drug, the latter can be administered at a much lower dosage while maintaining or exceeding its potency. Compositions containing the combination of highly tolerable compound and albumin-bind drugs are also disclosed.

### CROSS-REFERENCE TO RELATED APPLICATIONS

The present application claims the benefit of U.S. provisional application 60/657,427, filed Mar. 2, 2005, incorporated herein by reference.

### FIELD OF THE INVENTION

The invention relates in general to methods of increasing the safety and efficacy of albumin-binding drugs such as those used as anti-cancer, anti-infective or anti-hypertensive drugs, and in particular to a method of using Salus™ compounds, i.e., compounds that bind competitively with the albumin-binding drugs at the IB binding site of human serum albumin binding compounds in conjunction with the albumin-binding drugs so to increase the safety and efficacy of those drugs for their desired effects. By administering the Salus™ compound in an amount effective to competitively bind with the albumin-binding drug at the IB binding site, the efficacy of the drug will be increased substantially, and its safety will also be maximized because the drug will be effective at a much lower dosage. Compositions containing the combination of a Salus™ compound and albumin-binding drugs are also disclosed.

### BACKGROUND OF THE INVENTION

Human serum albumin (HSA) is the major protein of the circulatory system having plasma concentrations in the range from 30 to 50 mg/ml (approx. 0.6 mM). In addition to its principal role in the circulatory system, more than 40% of the total albumin is extravascular. It is a large protein of 66,500 MW being comprised of structurally homologous repeating domains, I, II and III, each in turn comprised of two subdomains (IA, IB, IIA, IIB, IIIA, IIIB). Albumin contributes 80% to colloidal osmotic blood pressure and to maintaining the pH of the blood, and possesses an exceptional capacity to bind and transport a plethora of biological and pharmaceutical compounds. Albumin, through its drug binding activity, is recognized as a major determinant of the adsorption, distribution, metabolism and excretion (ADME) of pharmaceuticals. Certain details regarding the atomic structure and the binding affinities of albumin and the specific regions primarily responsible for those binding properties have previously been disclosed, e.g., in U.S. patent application Ser. No. 08/448,196, filed May 25, 1993, now U.S. Pat. No. 5,780,594, U.S. patent application Ser. No. 08/984,176, filed Dec. 3, 1997, now U.S. Pat. No. 5,948,609, and U.S. patent application Ser. No. 10/506,043, having a US filing date of Apr. 5, 2005, and published as U.S. Pat. App. Pub. No. 2005/0182246 or WO 2003/074128, all of said references being incorporated herein by reference.

Due to the role of albumin in the bloodstream and its interaction with other drugs, it is important to understand the need to modulate the drugs interaction with human serum albumin because of the interplay between drug specificity to target and the various physiological processes such as metabolism, bioavailability, etc., all of which have previously affected the desired beneficial goal of achieving a therapeutic with the desired beneficial effect with minimal adverse effects. Recently, there have been some attempts to examine the relationship between albumin and certain drugs such as camptothecins, but such attempts have not focused on the particular binding region of human serum albumin and thus have not resulted in treatment regimens which could drastically improve the effectiveness of the particular drugs and the ability of patients to obtain effective treatment at smaller and more tolerable doses. Such research is reflected in articles such as J. Med. Chem. 1994, 37:40-46; J. Med. Chem. 2000, 43:3970-3980; Biochemistry 1994, 33, 10325-10336; Analytical Biochemistry 212:285-287 (1993) and US Pat. App. Pub. 2002/0193318, all of said articles incorporated herein by reference.

Thus, the important aspects of the interactions between drugs and HSA have not been taken into account in much of the drug design and development in the pharmaceutical field, and as a result, there are numerous cases of drugs that seem promising during animal trials but are failures when tried in human clinical trials. As has been noted by leading experts in the field of drug delivery, “An undesirable proportion of compounds with good biological activity fails to progress to later stages of drug development because of non-appropriate pharmacokinetic and pharmacodynamic properties . . . Out of the four aspects of pharmacokinetics (absorption, distribution, metabolism, excretion), distribution is the one that this protein (albumin) controls, because most drugs travel in plasma and reach their targets bound to this protein.” G. Colmenarejo, Medicinal Research Reviews, Vol 23, No. 3, 275-301, 2003. As a result of the failure to take into account the important properties of albumin, many millions of dollars of research have been wasted because drugs that seem promising during animal testing yet before administration in the human bloodstream have failed to take into account the particular properties of human serum albumin and how it may affect drug delivery in humans.

It is thus highly desirable to achieve a further understanding of the drug-albumin interactions and to be able to modulate those interactions for improving the drug development and delivery process. Such an approach is important because of the interplay between drug specificity to target and the various physiological processes such as metabolism, bioavailability, etc. all of which contribute to the desired beneficial goal of achieving a therapeutic with the desired beneficial effect with minimal adverse effects.

### SUMMARY OF THE INVENTION

It is thus an object of the present invention to provide Salus™ agents which can be administered along with a drug that binds to the IB site on human serum albumin and which can be utilized to increase the safety and effectiveness of that drug which can become much more effective at lower dosages.

It is further an object of the present invention to provide methods for maximizing the therapeutic effectiveness of drugs which bind to the IB site on human serum albumin by administering said drugs in the present of Salus agents which increase the safety and effectiveness of that drug which can become much more effective at lower dosages.

It is still further an object of the present invention to provide methods for treating diseases and conditions treated by drugs which bind to the IB site on human serum albumin, including conditions such as cancer, hypertension, infection, and numerous other treatments wherein effectiveness of the drug has been limited due to binding with albumin at the IB site.

These and other objects of the present application are obtained by virtue of the present invention which comprises a method of providing increased safety and efficacy during administration of albumin-binding drugs such as those used as anti-cancer, anti-infective, or anti-hypertensive drugs, or for numerous other conditions, via co-administration of a more tolerable compound that can competitively bind to albumin at the same site as the albumin-binding drug. In particular, the method of the present invention is directed to the modulation of the pharmacokinetics of those drugs which bind at the IB site on human serum albumin by co-administering a compound which has been designated as “Salus™” which is highly tolerable to humans and which can bind competitively with those albumin-binding drugs at the IB binding site so as to increase the safety and/or efficacy of the drug. The invention is advantageous in that by administering the highly tolerable Salus™ compound in a sufficient amount to compete with the targeted drug, the latter drug can be administered at a much lower dosage while maintaining or exceeding its potency. In addition, specific methods of maximizing the therapeutic effectiveness of drugs for particular applications are provided along with compositions containing the combination of highly tolerable Salus™ compound and drugs that bind at the IB region of HSA.

### BRIEF DESCRIPTION OF THE DRAWING FIGURES

The present invention is further illustrated in the drawings, wherein:

FIG. 1 is a schematic depiction of the opening of the lactone ring of camptothecin.

FIG. 2 is a stereo view of the difference map of Camptothecin in the binding site of human serum albumin FIG. 3 shows the percentage of active lactone form of Camptothecin in the presence of 30 mg/ml human serum albumin. After three hours, the level of active Camptothecin is 20% in the presence of the Salus™ agent in accordance with the invention (▴) versus essentially zero in the absence of the agent (▪).

FIG. 4 shows the percentage of active lactone form of 9-nitro-Camptothecin in the presence of 30 mg/ml human serum albumin.

FIG. 5 shows the percentage of active lactone form of 10-hydroxy-Camptothecin in the presence of 30 mg/ml human serum albumin.

FIG. 6 shows the free concentration of Teniposide in the presence of 30 mg/ml human serum albumin.

FIG. 7 shows the free concentration of Quinapril in the presence of 30 mg/ml human serum albumin.

FIG. 8 shows the availability of Sulfisoxazole in the presence of the Salus™ compound of the present invention.

FIGS. 9-13 show the improvement in the ability of anti-cancer drugs to kill breast cancer cells in the presence of Salus™ in accordance with the invention, including 10-hydroxy camptothecin (FIG. 9), Doxorubicin (FIG. 10); Epirubicin (FIG. 11), Topotecan (FIG. 12) and Teniposide (FIG. 13), wherein 301 represents the Salus™ agent.

### DETAILED DESCRIPTION OF THE PREFERRED EMBODIMENTS

In accordance with the invention, method and compositions are provided for increasing the effectiveness of drugs which bind with human serum albumin at the IB site which comprises administering a “Salus™” agent along with the drug which binds at the IB site. As set forth further below, a Salus™ agent is a compound which is highly tolerable in humans and which binds competitively with the albumin-binding drug at the IB binding site in such a manner as to maximize the therapeutic effectiveness of such a drug, i.e., it will be as effective or more effective at lower dosages, or will have increased effectiveness when used at its normal indicated dosage. As would be understood by one skilled in the art, therapeutic effectiveness will mean, improved pharmacology or pharmacokinetics, increased safety, an increase in the ability of the drug to treat the designed condition it has been administered to treat, etc., as determined by the normal parameters to assess effectiveness for a given treatment. For example, the maximized therapeutic effectiveness for an anti-cancer drug may be determined in terms of a reduction in the level of rapidly dividing cells, an anti-hypertensive would be more effective at reducing hypertension, an anti-infective might be measured in terms of effectiveness against a particular bacterial infection, etc., all parameters of which would be readily understandable to one of ordinary skill in the art and readily capable of detection and determination through conventional means used in a particular field.

Accordingly, in accordance with the invention, a Salus™ agent will be a compound that will bind competitively with the IB-binding drug and will be one that will generally be highly tolerable in the patient. In general, in addition to binding at the IB binding site on human serum albumin, there are four principal considerations for the selection of a preferred Salus™ agent, namely specificity, affinity, dosable plasma concentration, and therapeutic indication. With regard to specificity, it is preferred that they have specificity in binding location on human serum albumin which includes the albumin binding pocket at site IB. With regard to affinity, the agent should have reasonably high affinity for the IB binding pocket, and preferably will have greater affinity for the IB region that will the target therapeutic drug used in conjunction with the Salus™ agent which will be improved in terms of safety and/or efficacy. Generally compounds which have a K_{d} of 10⁵ or greater are highly preferred, however, lower K_{d}s of highly tolerated pharmaceuticals may be utilized where higher mM concentrations are achievable. It will also be understood that by Salus™ agent is meant that the Salus™ compounds of the present invention as described herein will include the physiologically acceptable salts and esters of the particular compounds described herein as well as the metabolic products or enantiomers of such compounds which also exhibit the properties of the compounds as described above.

With regard to dosage, it is desired to have an achievable dosable or blood concentration in the millimolar (mM) or greater range. Since albumin is present in the circulatory system at approximately 0.6 mM, it is preferably that the dosing should range from 0.1 mM to more than 23 mM. It is also preferable that the therapeutic indication of the Salus™ agent not interfere with the biological action the target therapeutic. If possible, the agent could be chosen to compliment that of the target therapeutic. Additional considerations include preferably a high therapeutic index, a history of safe use at the required effective dose and current FDA approval which will allow the Salus™ agent to be readily used in a variety of treatment regimens with presently available drugs which bind at the IB site on human serum albumin. In addition to currently used pharmaceuticals, agents can be selected from a group of compounds including currently approved pharmaceuticals, nutrients (including fatty acids and peptides), metabolic products and novel design compounds, preferably those that employ metabolic pathways which do not interfere with the pathway of the target drug being improved by the Salus™ agent. A list of examples of Salus™ agents useful in the present invention is provided in Table II below.

In accordance with the present invention, the Salus™ agents as set forth above will be utilized along with drugs that bind at the IB site by co-administering an effective amount of the Salus agent with the drug, including administration before, simultaneously with, or after administration of the IB-binding drug. As would be recognized by one of ordinary skill in the art, the amount of any Salus agent necessary to achieve the maximum therapeutic effectiveness of a target drug will vary depending on the size and condition of the patient as well as the particular agent and drug chosen, and it will be readily understood that such an effective amount would be determined by the appropriate physician or other health care professional based on the circumstances of the treatment. The effective amount of the agent used along with the particular IB-binding drug will this vary from patient to patient, and will be that amount needed to obtain improvement in the safety and/or effectiveness of the drug in treating the condition it is designed to treat as set forth above.

In this regard, the IB-binding drugs usable in the present invention will be those drugs which specifically bind at the IB region of human serum albumin and which are used to treat a variety of illnesses and conditions in the patient. These drugs will also preferably be ones that are compatible with a given Salus agent, and will generally have a lower affinity to the IB region than the Salus agent it will be used with. A non-limiting list of drugs which bind with albumin at the IB site and will be usable in the present invention is provided herewith in Table I below.

Accordingly, the Salus agent of the present invention will be used with a variety of IB-binding drugs for which it competitively binds at the IB site, and it is generally preferred that the Salus agent has a higher affinity to that site than the IB-binding drugs. In this regard, it is generally preferred that the Salus agent will also be able to block or displace the IB-binding drug from the IB site in human serum albumin when these compounds are in the bloodstream. The present invention thus contemplates compositions comprising an effective amount of the Salus agent in combination with a IB-binding drug which will generally be used at an amount of at or below its normal dosage. When desired, the Salus agent and the IB-binding drug may be administered together in a single unit, and such compositions will generally include a physiologically acceptable vehicle, carrier or excipient as generally known in the art.

For example, the Salus agent may be used by co-administering the agent with a therapeutic IB-binding drug used as an anti-cancer drug, e.g., one that reduces the level of rapidly dividing cells in a patient in need thereof, so that an effective composition in accordance with the invention may comprise a drug that reduces the level of rapidly dividing cells in a patient and that binds to human serum albumin at the IB site and a compound that competitively binds to human serum albumin in an amount effective to increase the free concentration of the drug in the bloodstream of a patient in need. As indicated herein, suitable Salus agents may include clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate, and a suitable anticancer drug binding at the IB site may include the Camptothecin family of drugs, including but not limited to Camptothecin, 10-hydroxycamptothecin, 9-aminocamptothecin, Topotecan and Irrinotecan, the anthracyclin family of drugs including but not limited to Doxorubicin and Epirubicin, the Taxol family of drugs including but not limited to Paclitaxol, the Etoposide family of drugs and the Teniposide family of drugs. Other specific compounds or drugs with the properties as set forth above will be readily determined by one of ordinary skill in the art and these will fall within the scope of the present invention as well.

In accordance with the present method, the therapeutic effectiveness of a drug that binds at the IB site on human serum albumin will be enhanced by co-administering that drug with an effective amount of a compound that is highly tolerable in patients which is also competitive with the IB-binding drug at the IB site, and the IB-binding drug will be made more effective by virtue of the competitive compound which will generally have more affinity to the IB site than the IB-binding drug and will generally be able to block or displace the IB-binding drug from that site. The IB site on human serum albumin is well known and would be readily understood by one skilled in the art, and this site has been mapped out along the HSA sequence, such as in one or more of the patents identified above. Table I below provides a listing of the drugs known to bind at the IB site, and the present invention will be useful in enhancing the effect of the IB-binding drugs by making them more effective at current dosages or by allowing the same effective strength of the particular drug when used at a lower level. As indicated above, such treatment methods will be particular useful for patients who have a hard time tolerating drugs, in situations wherein a particular drug may be cytotoxic at high doses, or in those cases wherein a drug must be administered over a long period of time.

As indicated in the chart at Table I and as set forth herein, one particular application of the Salus™ agents of the present invention is with regard to anti-cancer drugs. In this regard, there are a number of anti-cancer drugs that bind specifically to the IB region of human serum albumin, and such drugs can be combined with the Salus™ agents of the present invention to become safer and more effective in that a lower amount of the drug can have the same effect as the administration of the drug at its normal dosage, but not in the presence of a Salus™ agent. Included in the anti-cancer drugs which can be used in the present invention would be the Camptothecin family of drugs, including but not limited to Camptothecin, 10-hydroxycamptothecin, 9-aminocamptothecin, Topotecan and Irrinotecan, the anthracyclin family of drugs including but not limited to Doxorubicin and Epirubicin, the Taxol family of drugs including but not limited to Paclitaxol, the Etoposide family of drugs and the Teniposide family of drugs. In such a case, wherein the drug is an anticancer drug, there would be a number of ways of determining to maximize the therapeutic effectiveness of the present Salus™ agent, including, e.g., the reduction of the level of rapidly dividing cells, an increase in the therapeutic index of the IB-binding drug when administered with a Salus™ agent, an increase in the free concentration of the IB-binding drug, improved pharmacology or pharmacokinetics, increased safety, etc.

Accordingly, in these aspects of the invention, the competitive Salus™ compound will be administered in an amount effective to cause an increase in the therapeutic benefits afforded by the IB-binding drug. These drugs and competitive compounds can be taken in any suitable form used by practitioners skilled in the art of healthcare, including administration orally, intravenously, parenterally, or any of a number of other ways commonly used to deliver drugs and other agents intended for internal use. As indicated above, a number of suitable Salus™ agents can be used with the IB-binding anti-cancer drugs in accordance with the invention, including for example clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate.

In a typical example, the competitive Salus™ compound can be employed at such a level so that it reaches a plasma concentration in patient in the range of about 0.1 mM to 25.0 mM, and its application can occur before, simultaneously with, or after the administration of the IB-binding drug, provided that the drug and competitive compound will be found in the bloodstream at the same time. It is even further the case that a Salus compound may also aid in therapeutic effectiveness by itself having the same therapeutic effects on a particular disease or condition as the IB-binding drug it will be administered with. For example, it is possible that the Salus compound introduced with the anticancer drug itself has anti-cancer properties, i.e., it can lead to a reduction in the size or number of tumors, it can reduce the level of rapidly dividing cells in a patient, and/or can other effects such as increasing the therapeutic index of the IB-binding drug, increase its free concentration in the bloodstream, etc. Accordingly, in one aspect of the present invention, a method is provided for increasing the free concentration of a drug that reduces the level of rapidly dividing cells in a patient in need thereof and that binds at the IB site of human serum albumin comprising administering the drug in the presence of a compound that binds competitively with said drug at the IB site of human serum albumin in an amount effective to increase the free concentration of said drug in the bloodstream of the patient.

As indicated above, in addition to methods wherein the IB-binding drug is co-administered with the Salus compound, it is possible to provide compositions which reflect the combination of the Salus agent with the therapeutic drug such as an anticancer drug. In this case, the composition of the invention will be capable of reducing the level of rapidly dividing cells in a patient in need thereof, and will comprise a drug that reduces the level of rapidly dividing cells in a patient and that binds to human serum albumin at the IB site and a compound that competitively binds to human serum albumin in an amount effective to increase the free concentration of the drug in the bloodstream of a patient in need. The nature of the Salus agents and anticancer drugs useful in the compositions of the present invention are described above. Such compositions will generally also include conventional ingredients common to drug forms such as a pharmaceutically acceptable vehicle, carrier or excipient.

Similarly, many other types of drugs which bind at the IB region of human serum albumin can be improved in terms of safety and/or efficacy by being administered along with the Salus agents referred to above, such as by co-administration methods or in compositions wherein the effective amount of the Salus agent is added to a given dosage of the IB-binding drug. For example, in accordance with the present invention, a method for increasing the effectiveness of a drug that reduces hypertension is provided which comprises administering an anti-hypertensive drug that binds to human serum albumin at the IB site along with a compound that binds competitively with said drug at the IB site of human serum albumin in an amount effective to manage the reduction of hypertension in the patient. Suitable Salus agents for use in this method are those as set forth above, including agents such as those included in Table II below, and the antihypertensive can be any suitable anti-hypertensive drug that binds at the IB region. Included in the drugs that are suitable for use in the present invention are Prazosin, Ramapril, Quinapril, Terazosin, Hydralazine, Methyldopate. Valsartan, Irbesartan, Alprenolol, Chlorothiazide and Doxazosin. The effect of the Salus drug on the anti-hypertensive is generally one that will modulate the free concentration of the anti-hypertensive drug in the patient and allow it to be more effective in reducing hypertension.

Still another type of drug which will be improved in terms of safety and effectiveness will be those anti-infective drugs which bind to human serum albumin at the IB region which will exhibit increases in their ability to reduce or eliminate bacterial, fungal or other infections in a patient through use of the present invention. Accordingly, in accordance with this aspect of the invention, a method for increasing the effectiveness of an anti-infective drug is provided which comprises administering an anti-infective drug that binds to human serum albumin at the IB site along with a compound that binds competitively with said drug at the IB site of human serum albumin in an amount effective to maximize the therapeutic effectiveness of the anti-infective drug, e.g., it reduces or eliminates infections in the patient. Suitable Salus agents for use in this method are those as set forth above, including agents such as those included in Table II below, and the anti-infective can be any suitable anti-infective drug that binds albumin at the IB region. Included in the drugs that are suitable for use in this aspect of the invention are Sulfisoxazole and Cefamandole Nafate, and the Salus agents useful with the anti-infective drugs of the invention will generally be those that will modulate the free concentration of the anti-infective drug in the patient and allow it to be more effective in reducing or eliminating infection.

In short, the Salus™ agents of the present invention can be useful in improving the therapeutic properties of drugs that bind to the IB region of human serum albumin, and indeed as shown below, the therapeutic index (TI) for every agent tested against an anticancer drug that binds to IB was increased, in many cases substantially. In addition, the Salus agent of the invention will tend to improve the free drug concentration of the IB-binding drug used in the invention, and this improved efficacy and has implications for new therapeutic indications, for example higher available free drug concentration may facilitate the availability of the drug through certain organ circulatory interfaces, notably the brain. The Salus agents of the invention will also improve the safety of IB-binding drugs, especially highly cytotoxic drugs such as anti-cancer and anti-infectives by lowering the effective dose with concomitant increase in efficacy so that the drug will be better tolerated by the patient, or be able to be administered over a longer period of time without side effects related to cytotoxicity in the patient. This will be particularly effective as an alternate mode of treatment for refractory cases or for individuals whose health will not tolerate the traditional dosing.

Accordingly, the ability to tune the pharmacokinetics of drugs by the deliberate and knowledgeable use of the Salus agents of the present invention which modulates the pharmacokinetic of the target compound or compounds (or possibly both drugs if both activities are of desired therapeutic benefit) will be immensely important in many avenues of pharmaceutical therapy, including those dealing with highly cytotoxic drugs, those used in oncology, or those used for anti-infective purposes which may normally be difficult for patients to tolerate.

In general, the present invention can provide a method for increasing or maximizing the therapeutic effectiveness of a drug, such as those which reduce the level of rapidly dividing cells in a patient in need thereof and which binds human serum albumin and the binding of which is affected/reduced by a Salus™ compound that binds to albumin at IB site. The Salus™ IB-binding compound can influence the said drug's binding to albumin in two possible ways. Firstly, since the drug also binds albumin at IB site, the Salus™ compound binds albumin competitively with the drug; or by the binding of the Salus™ compound to albumin at IB site, albumin under goes conformational change such that the said drug's binding is affected through allosteric effect which may result in a reduction in affinity to other binding sites. By co-administering the Salus™ compound in an effective amount in accordance with the invention, these effects on the binding of the IB-binding drug will allow the present invention to maximize the therapeutic effectiveness of said drug in the patient.

While the invention has been described above with regard to preferred embodiments, it is clear to one skilled in the art that there will be additional embodiments, compositions and methods which fall within the scope of the invention which have not been specifically described above.

The following examples are provided which exemplify aspects of the preferred embodiments of the present invention. However, it will be appreciated by those of skill in the art that the techniques disclosed in the example which follow represent techniques discovered by the inventors to function well in the practice of the invention, and thus can be considered to constitute preferred modes for its practice. Moreover, those of skill in the art will also appreciate that in light of the present specification, many changes can be made in the specific embodiments which are disclosed and still obtain a like or similar result without departing from the spirit and scope of the invention

### EXAMPLE I

### Salus™ Drug Formulation in Accordance with the Invention

An important application of the CADEX™ knowledge base is the launch of Salus™ drug formulation. We have now demonstrated that the therapeutic profile of at least several problematic drugs can be dramatically improved by co-administering a specially selected secondary compound which modulates the albumin-related pharmacokinetics of the first. This therapeutic drug displacement approach is based on the precise identification of the drug interaction and careful selection of displacing agent with the proper attributes. (These drug interactions have been previously uniquely determined by NCP scientists after a multi-year multimillion dollar survey of the atomic structures. These IB-binding drugs are identified in Table 1 herein). The identified drug combinations, the basis for the NCP Salus™ (Salus: Latin for “safety” “wholeness” “salvation”) drug formulation, offer the following advantages:
precise binding modulation/displacement
high effectiveness with lower required dosage
utilizes FDA approved pharmaceuticals
improves tolerance and safety by lowering dose requirements for many toxic therapeutics
broadly applicable in all therapeutic areas
improves efficacy of many new and existing pharmaceuticals that are highly bound to serum albumin
in many cases rapid market entry and approval are possible

After review of IB binding therapeutics, we selected an appropriate Salus candidate with the attributes of: 1) high affinity to IB; 2) low toxicity and safe history as pharmaceutical; 3) therapeutic indication of the Salus which is unlikely to interfere with the applications; and 4) achievable, convenient and FDA approved dosing to improve pharmacokinetics. From these criteria we chose the lipid lowering drug Clofibrate as a likely Salus candidate to improve the drug pharmacokinetics of the target therapeutics. Since the projects early history involved the chemistry of the camptothecin family of anticancer drugs, we describe below the initial test.

Camptothecin Analogs: Drug Efficacy & Safety Opportunity

Camptothecin, an alkaloid compounds derived from plants, was found to have anti-cancer activity in 1960s effective in more than 13 human cancer xenografts lines carried by immunodeficient (nude) mice (2,3). As a result of its potent activity against cancer cells, it was rushed into clinical trials shortly after its discovery. However, all trials were soon terminated due to very disappointing results in humans in spite of excellent activity against tumor cells in xenografts (4-6). Recently, interests on Camptothecin have resumed after it was identified as an inhibitor of Topoisomerase I by forming covalent complex with DNA and Topoisomerase. This category of compounds are attractive because of their specific toxicity against cells undergoing DNA replication, a process cancer cells are going through much more frequently than normal cells.

The main reason of its inactivity in humans was found to be related to human serum albumin in blood. Generally, Camptothecin and its derivatives have two forms, the open carboxylate (inactive) and the closed lactone (active) form ( ). At pH above 7.0, the two forms exist at 50:50 in aqueous solutions. In whole blood, human albumin binds preferentially to the carboxylate form at a binding affinity of ˜10⁶ M⁻¹, which rapidly diminishes the available lactone form from the blood stream (7-9). On the other hand, mouse albumin has reduced affinity to these compounds resulting in active concentrations of approximately 50%. The differences in free concentration between the mouse and human correlate directly with the observation that Camptothecin and derivatives have been shown to eliminate all human cancer cells introduced in nude mice.

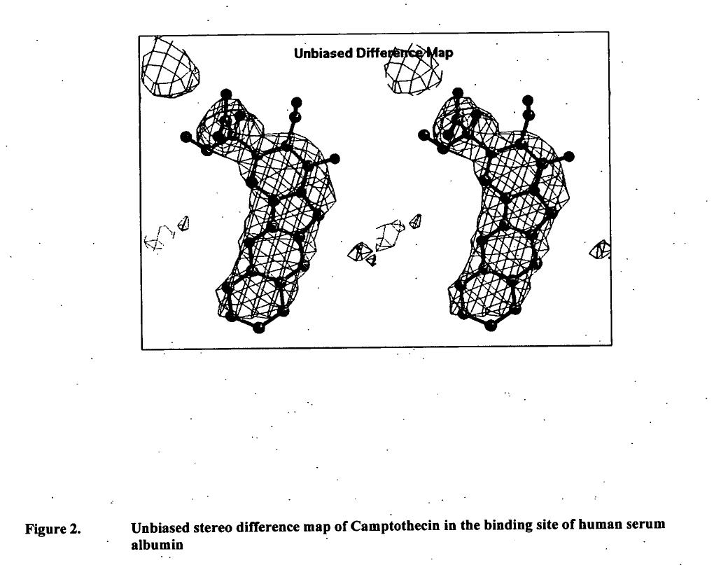

Using our CADEX™ technology, we have determined the x-ray structure of Human serum albumin complexed with Camptothecin and several derivatives. The unbiased difference density clearly indicated the open carboxylate form in the binding site (FIG. 2). The detailed interactions between residues and Camptothecin illuminated the selectivity of the two different forms (proprietary) and the enhanced activity in the mouse.

Salus™ Drug Combinatorial Agents were identified using the CADEX knowledge base. Select Salus™ agents have been shown to competitively inhibit the albumin binding of Camptothecin and several of its derivatives resulting in a higher free concentration of the active component in the blood. For example, in the presence of a selected agent, the percentage of the active lactone form of Camptothecin significantly increased, in solution containing 30 mg/ml human serum albumin (FIG. 3) and in plasma or whole blood (data not shown), remaining at a level (20%) much higher than Topotecan (12%)—an FDA-approved anticancer drug. Equally dramatic results achieved with 9-nitro-Camptothecin and 10-hydroxy-Camptothecin indicate that the selected Salus agent is generally applicable to the Camptothecin family. In a separate study, and in further validation of therapeutic value, the increase in human plasma of active form directly correlates with increased inhibition of Topoisomerase I in vitro.

The example shown below not only validates the power of the Salus™ technology, but has tremendous implications in clinical cancer therapy. Camptothecin, being toxic only to the cells in S-phase, must be exposed to the cancer cells over a prolonged period of time at high level to be effective. Our findings clearly show that the co-administration of Salus™ agent(s) highly bound to the identified Camptothecin site, can achieve dramatically enhanced therapeutic profiles for this family of drugs. Since the preferred Salus™ agent represents a highly safe FDA approved pharmaceutical, clinical studies could be accelerated.

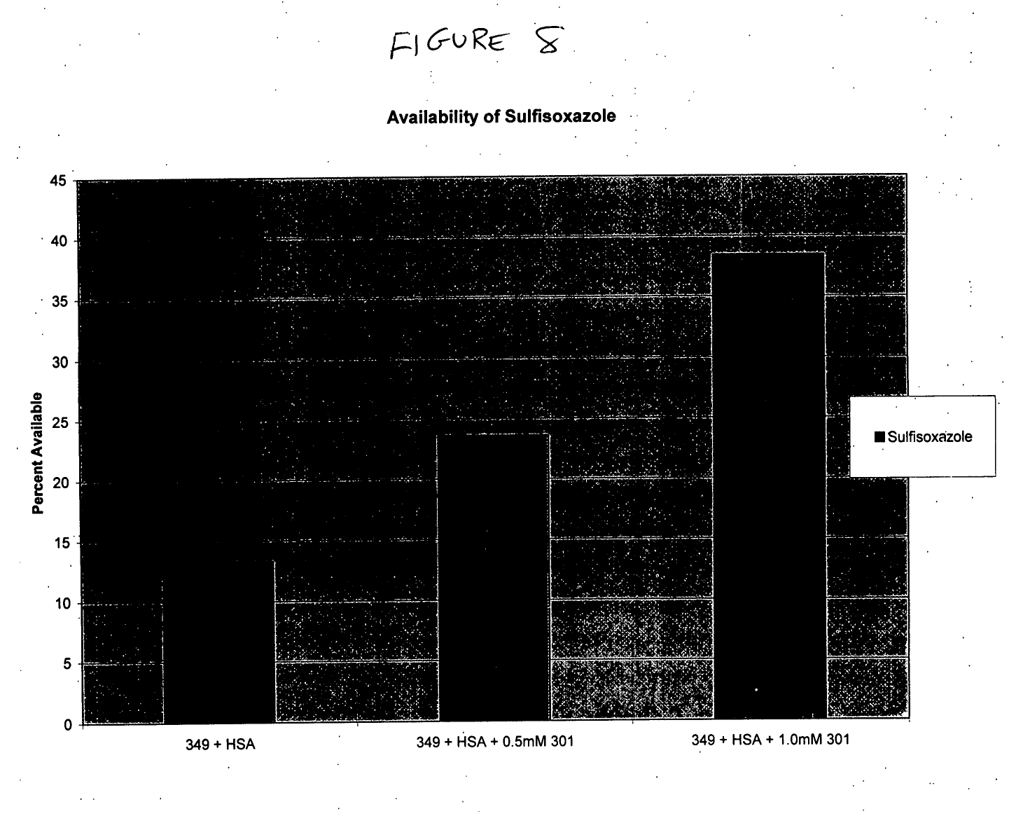

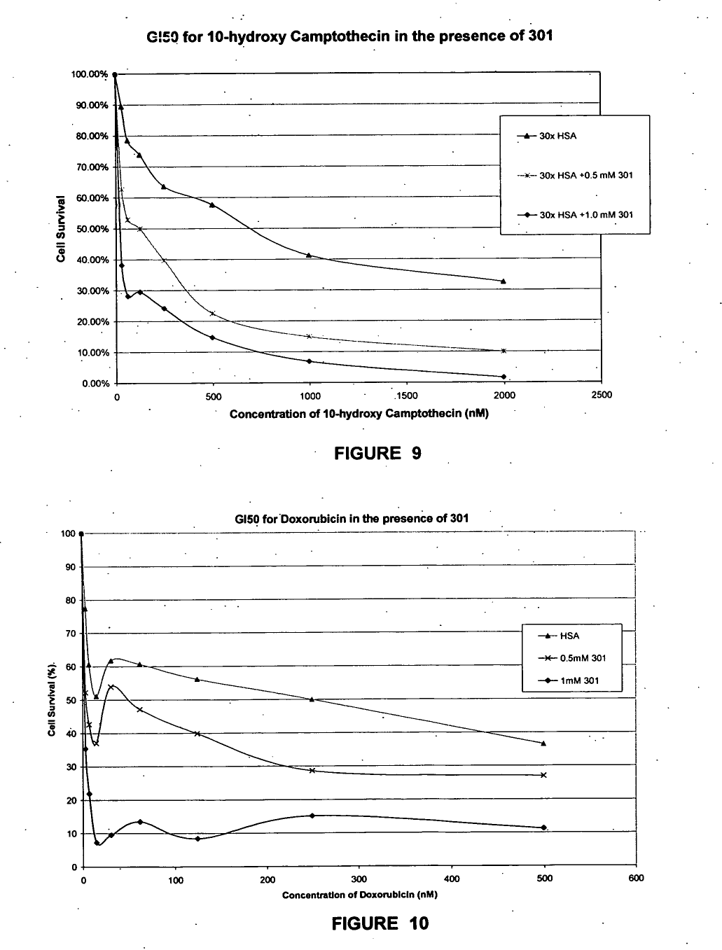

To further validate the concept in vitro studies using human breast cell carcinoma line and clinically approved blood concentrations of both clofibrate (301) and 10-hydroxycamptothecin were performed. FIG. 9 graphs the results GI 50 below. This graph shows approximately 16 fold improvement in the GI 50 values. Additionally at clinically approved concentrations of both drugs in the presence of blood concentrations of human serum albumin, without Salus, approximately 33% of the cells were surviving at the end of the test vs. approximately 2%.

Determinations of the Therapeutic Index Based on In Vitro Cell Assays

Therapeutic index is defined as the ratio of the median lethal dose (LD₅₀) to the median effective dose (ED₅₀). Recently, Dr. Björn Ekwall and colleagues in the Multicenter Evaluation of In-Vitro cytotoxicity trial based in Uppsala, Sweden demonstrated that the non-animal, human cell line tests are more predictive of human toxicity than in vivo (animal) data. While animal tests are at best only about 65 percent predictive of human acute toxicity, a combination of four cell tests can determine of a substance is dangerous to humans with better than 80 percent precision.

We have decided to use the human MRC-5 lung fibroblast cell line to test IC₅₀ of 10-hydroxycamptothecin (10-HC) in the absence and presence of Salus™ 301. The ED₅₀ values were determined with human MDA-MB-435S breast carcinoma cell line.

The following table listed the results from the best case scenario:

| Experiments | ED₅₀ (nM) | IC₅₀ (nM) | Therapeutic Index |
|---|---|---|---|
| 10-HC without 301 | 698.4 | 6782.6 | 9.7 |
| 10-HC with 0.5 mM 301 | 126.98 | 6000.0 | 47.3 |
| 10-HC with 1.0 mM 301 | 23.81 | 3304.3 | 138.8 |

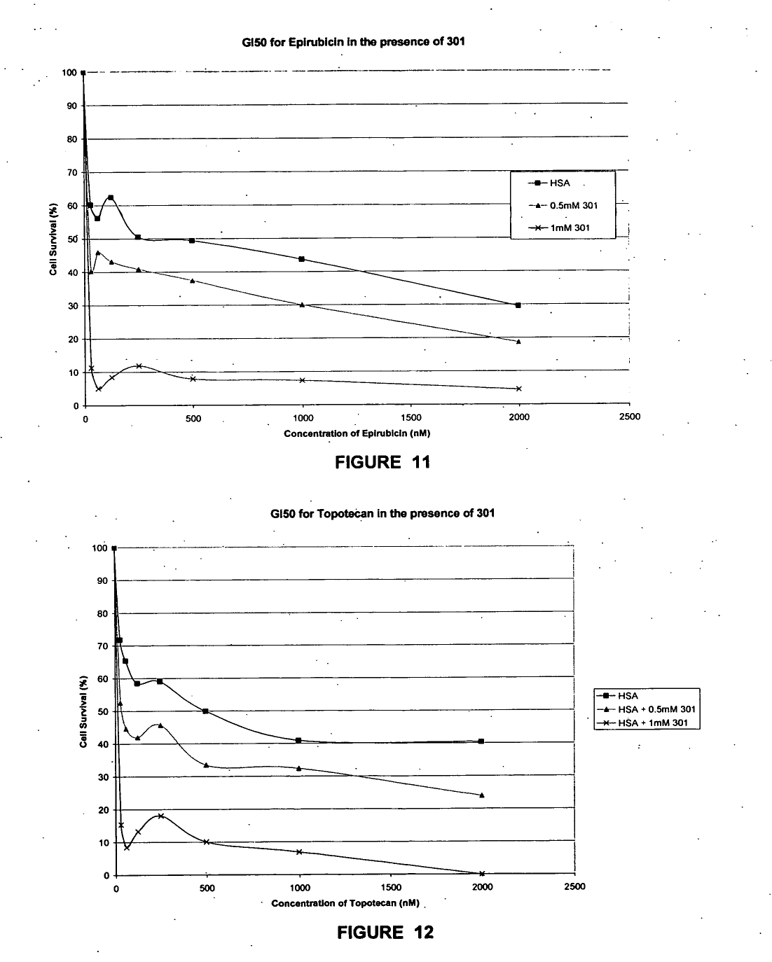

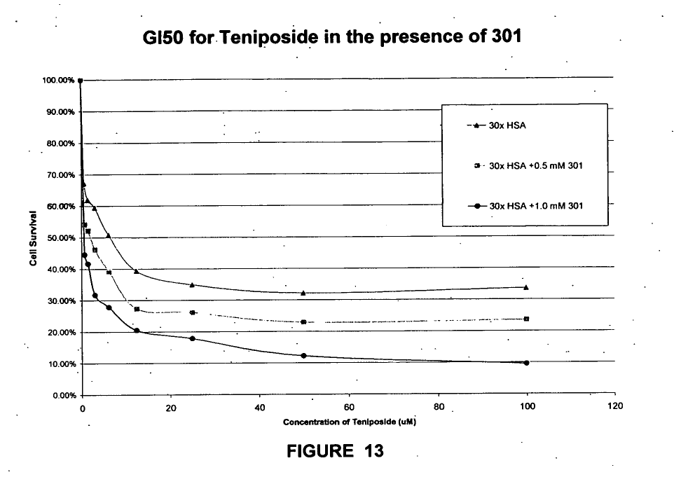

The Salus principle has been demonstrated with several anticancer and anti-infective drugs. In vitro activities against human breast cell carcinomas are shown in FIGS. 9-13 herein. In each case, there was marked improvement in the anti-cancer activity using the Salus technology. These tests included in vitro studies for anticancer therapeutics including Camptothecin, 10-hydroxycamptothecin, Topotecan, Irrinotecan, Etopiside*, Doxorubicin, Epirubicin, Teniposide, and Paclitaxol* (with * indicating that some assays are still in progress).

In general, the Salus agents of the present invention can be useful in improving the therapeutic index (TI) for every agent tested against an anticancer drug that binds to IB. The TI improvements can be substantial, including increase up to 10 to 30 times. In addition, the Salus agent of the invention will tend to improve the free drug concentration of the IB-binding drug used in the invention, and this improved efficacy and has implications for new therapeutic indications, for example higher free drug concentration available may facilitate the availability of the drug through certain organ circulatory interfaces, notably the brain. The Salus agents of the invention will also improve the safety of a drug, especially highly cytotoxic drugs such as anti-cancer and anti-infectives by lowering the effective dose with concomitant increase in efficacy so that the drug will be better tolerated by the patient, or be able to be administered over a longer period of time without side effects related to cytotoxicity in the patient. This will be particularly effective as an alternate mode of treatment for refractory cases or for individuals whose health will not tolerate the traditional dosing.

Accordingly, the ability to tune the pharmacokinetics of drugs by the deliberate and knowledgeable use of the Salus agents of the present invention which modulates the pharmacokinetic of the target compound (or possibly both drugs if both activities are of desired therapeutic benefit) will be immensely important in many avenues of pharmaceutical therapy, including those dealing with highly cytotoxic drugs, those used in oncology, or those used for anti-infective purposes which may normally be difficult for patients to tolerate.

The following references referred to above are incorporated in this application as if set forth herein in their entirety:

1. Horton, J. and Bushwick, B. 1999. Am. Family Physician 59: 635-647.
2. Wall, M. E., Wani, M. C., Cook, C. E., et al. 1966. J. Am. Chem. Soc. 88: 3888-3890.
3. Dewys, W. D., Humphreys, S. R., and Goldin, A. 1968. Cancer Chemother. Rep. 52: 229-242.
4. Gottlieb, J. A. and Luce, J. K. 1972. Cancer Chemother. Rep. Part I 56: 515-521.
5. Muggia, F. M. Creaven, P. J., Hanson, H. H., et al. 1972. Cancer Chemother. Rep. Part 156: 515-521.
6. Moertel, C. G., Schutt, A. J., Reitemerer, R. C., and Hahn, R. G. 1972. Cancer Chemother. Rep. Part 156: 95.
7. Giovanella, B. C., Stehlin, J. S., Wall, M. E., et al. 1989. Science 246: 1046-1048.
8. Mi, Z. and Burke, T. G. 1994. Biochemistry 33: 10325-10336.
9. Mi, Z. and Burke, T. G. 1994. Biochemistry 33: 12540-12545.

### EXAMPLE 2

### Additional Studies Regarding Salus™ Drug Formulation in Accordance with the Invention

Camptothecin (CPT) has been shown to inhibit the growth of a variety of animal and human tumors. Camptothecin and its related congeners display a unique mechanism of action: they stabilize the covalent binding of the enzyme topoisomerase I (topo I), an intranuclear enzyme that is overexpressed in a variety of tumor lines, to DNA. This drug/enzyme/DNA complex leads to reversible, single strand nicks that, according to the fork collision model, are converted to irreversible and lethal double strand DNA breaks during replication. Therefore, due to the mechanism of its cytotoxicity, CPT is S-phase specific, indicating that it is only toxic to cells that are undergoing DNA synthesis. Rapidly replicating cells like cancerous cells spend more time in the S-phase relative to healthy tissues. Thus, the over expression of topo I combined with the faster rate of cell replication, provide a basis through which camptothecins can selectively affect the cytoxicity of cancerous cells rather than healthy host tissues. It is important to note that due to the S-phase specificity of the camptothecins, optimal inhibition of topo I requires continuous exposure to the camptothecin agent.

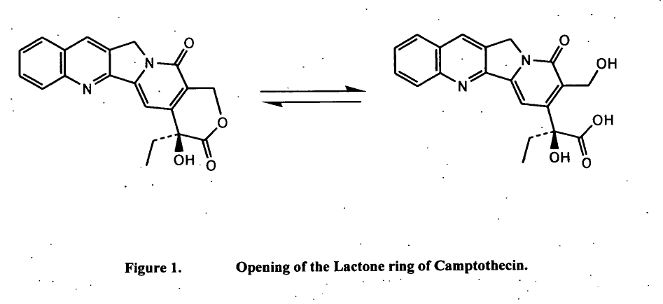

A closed alpha-hydroxy lactone (E) ring of CPT is an essential structural feature. An intact ring is necessary for the diffusion of the electroneutral form of the drug across membrane barriers and into cells by passive transport and, directly relevant to its in vivo anti-tumor potency, is required for the successful interaction of CPT with the topoisomerase I target. This essential lactone pharmacophore hydrolyzes under physiological conditions (pH 7 or above) and, therefore, the drug can exist in two distinct forms: 1) the biologically active, ring-closed lactone form; and 2) the biologically-inactive, ring-open carboxylate form of the parent drug (FIG. 1).

Unfortunately, under physiological conditions the drug equilibrium favors hydrolysis and, accordingly, the carboxylate form of the camptothecin drug persists. The labile nature of this alpha-hydroxy lactone pharmacophore has significantly compromised the clinical utility of the camptothecins, as continuous exposures to the active lactone form are requisite for efficacy purposes.

In human blood and tissues, the camptothecins exist in an equilibrium of active lactone form vs. inactive carboxylate form and the directionality of this equilibrium can be greatly affected by the presence of human serum albumin (HSA). Time-resolved fluorescence spectroscopic measurements taken on the intensely fluorescent camptothecin lactone and camptothecin carboxylate species have provided direct information on the differential nature of these interactions with HSA. The lactone form of camptothecin binds to HSA with moderate affinity yet the carboxylate form of camptothecin binds tightly to HSA, displaying a 150-fold enhancement in its affinity for this highly abundant serum protein. Thus, when the lactone form of camptothecin is added to a solution containing HSA, the preferential binding of the carboxylate form to HSA drives the chemical equilibrium to the right, resulting in the lactone ring hydrolyzing more rapidly and completely than when camptothecin is in an aqueous solution without HSA. In turn, this effect has negatively impacted the topoisomerase I inhibitory activity of many camptothecins and, by extension, negatively affects their clinical utility.

The important role that HSA plays in the stability of the camptothecins varies relative to drug structure. For drugs such as camptothecin and 9-aminocamptothecin, HSA functions as a biological sink for the carboxylate form. As a result, in whole human blood, 5.3% of camptothecin and only 0.5% of 9-aminocamptothecin remain in the lactone form at equilibrium. In contrast, A, B-ring substitutions of CPT, specifically at the 7- and 10-positions, can inhibit the preferential binding interactions between the camptothecin carboxylate and HSA. Accordingly, camptothecin congeners such as topotecan and SN-38, the biologically active form of the prodrug CPT-11, display lactone levels at equilibrium of 11.9% and 19.5%, respectively. Ultimately, by modulating the circulatory and tissue levels of free and active camptothecin drug, HSA can negatively impact the anti-cancer efficacy of the camptothecin agent.

The effect of serum albumins on camptothecins also differs markedly between lower vertebrates and humans and this variance has obscured the judicious selection of analogs for advancement to clinical trials. These interspecies differences have lead to significant anomalies when the data from animal models and clinical studies are compared. In particular, 9-aminocamptothecin has displayed striking activity in murine models bearing brain tumors. However, the pharmacokinetics of 9-aminocamptothecin in mice are quite different from those in humans; notably, 9-aminocamptothecin lactone levels are approximately 100-fold higher in murine blood relative to human blood. This discrepancy is due to the reduced binding of the carboxylate form of 9-aminocamptothecin to murine albumin. The logical extension of this finding is that approximately 100-fold more free lactone, which is able to cross cell membranes or the blood-brain barrier, is present in the mouse than it is in humans. The clinical relevance of this interspecies variation is underscored by a recent trial: 99 brain cancer patients were treated intravenously with 9-aminocamptothecin; the therapy was grossly ineffective (one partial responder) due to the likelihood that 99.5% of the drug was in the carboxylate form, bound to HSA and unable to transverse the blood-brain barrier.

The inherent blood instability of camptothecin has resulted in an extensive research effort to surmount the problem. Efforts to realize a blood stable camptothecin agent with potent anti-tumor activity have been primarily focused on formulation, such as liposomal preparations of the drug, and rational drug design, such as the development of the class of beta-hydroxy lactone camptothecins known as the homocamptothecins. The work described herein describes a third approach to maintaining a potent and more blood stable camptothecin congener: the modulation of camptothecin drug binding to HSA by implementing competing molecules that also bind HSA.

The camptothecins are not unique in their ability to bind albumin, as a variety of small molecules interact with this protein. A relatively large protein, 67 kD, albumin is distributed both in the plasma and in the interstitial fluid. Being one of the most abundant plasma proteins, its circulatory level ranges from 35 to 50 mg/ml (approximately 0.6 mM). The principal biological function of HSA is to maintain colloid osmotic pressure in the vascular system and to transport fatty acids and bilirubin. However, by hydrophobic and/or ionic interactions, a variety of small molecules bind tightly to albumin. Electroneutral and basic drugs may bind to albumin by hydrophobic binding interactions, and, as albumin has a net cationic charge, anionic drugs bind avidly to albumin via electrostatic interactions. Recent x-ray crystallography and competition data obtained by the present inventors reveal that camptothecin carboxylate preferentially associates with a newly characterized drug binding site in subdomain IB which has been identified in previous applications as a dominant binding site for many of pharmaceuticals.

The ability of human serum albumin to avidly bind to a variety of small molecules offers the possibility of competitively attenuating the negative effects human serum albumin on the in vivo anti-cancer and/or anti-HIV activity of camptothecin compounds and numerous other compounds that have high binding affinity for human serum albumin. This therapeutic drug displacement approach is based on the precise identification of the drug interaction and careful selection of displacing agent with the proper attributes. The identified drug combinations, the basis for the NCP Salus™ drug formulation using the Salus agents as discussed above will thus be useful in improving the performance of these drug formulations. This is critical because no prior methods have recognized or attempted to deal with the problem caused by the human serum albumin binding activity, and thus methods and compositions are needed which can attenuate the negative effects of human serum albumin on the stability of compounds such as camptothecin compounds, e.g., camptothecin or 9-aminocamptothecin, and other compounds or drugs, such as anti-cancer and ACE inhibitors, which have a high affinity for human serum albumin.

X-ray structure studies by the inventors have identified Camptothecin to bind in a distinct pocket within IB subdomain of human serum albumin. The unbiased difference density clearly indicated the open carboxylate form in the binding site (FIG. 2).

The detailed interactions between residues and Camptothecin illuminated the selectivity of the two different forms and the enhanced activity in the mouse. This discovery offers the method and formulation to select other compounds that bind in the same site to competitively inhibit the albumin binding of Camptothecin and several of its derivatives resulting in a higher free concentration of the active component in the blood.

Select Salus™ agents have been shown to competitively inhibit the albumin binding of Camptothecin, several of its derivatives, and other therapeutics, resulting in a higher free concentration of the active component in the blood. The precise determination of ligand binding location on the structure of human serum albumin has been worked out at atomic detail for a very large number of pharmaceuticals and ligands as previously disclosed in prior provisionals and applications. Some of the preferred high affinity Salus IB displacing agents can include bicalutamide, clofibrate, glipizide, ramipril, and teniposide.

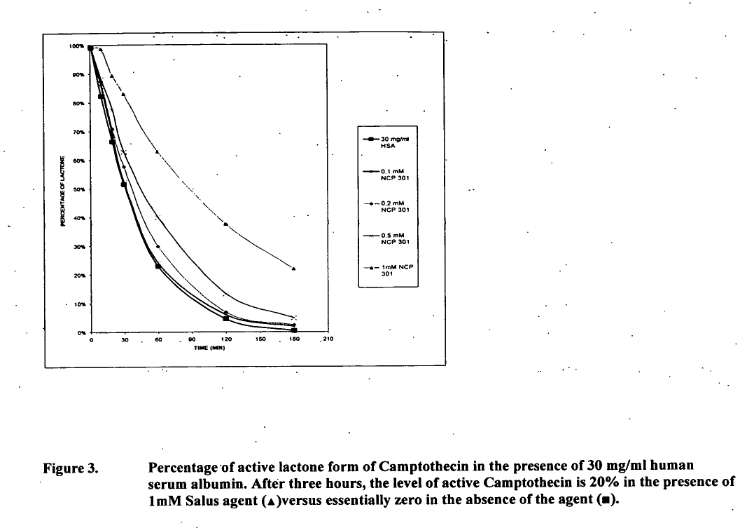

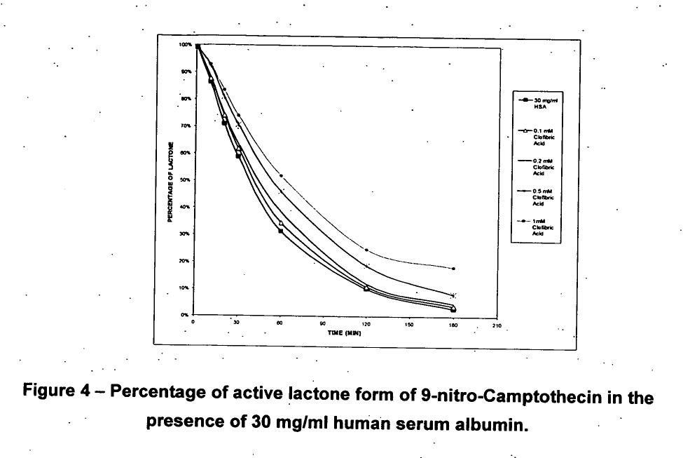

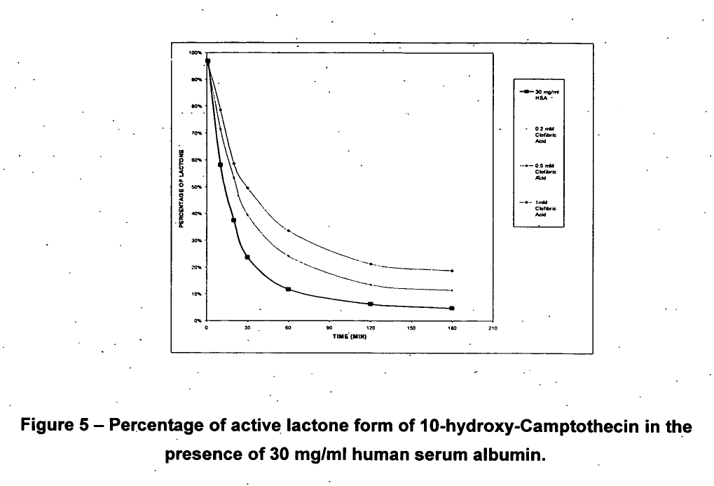

For example, in the presence of the Salus agent identified by the present inventors to bind in the same IB site of human serum albumin with a high binding constant and high therapeutic dosage, the percentage of the active lactone form of Camptothecin significantly increased, in solution containing 30 mg/ml human serum albumin (FIG. 3), remaining at a level (20%) much higher than Topotecan (12%)—an FDA-approved anticancer drug. Equally dramatic results achieved with 9-nitro-Camptothecin (FIG. 4) and 10-hydroxy-Camptothecin (FIG. 5) indicate that the selected Salus™ agent is generally applicable to the Camptothecin family. In a separate study, and in further validation of therapeutic value, the increase in human plasma of active form directly correlates with increased inhibition of Topoisomerase I in vitro.

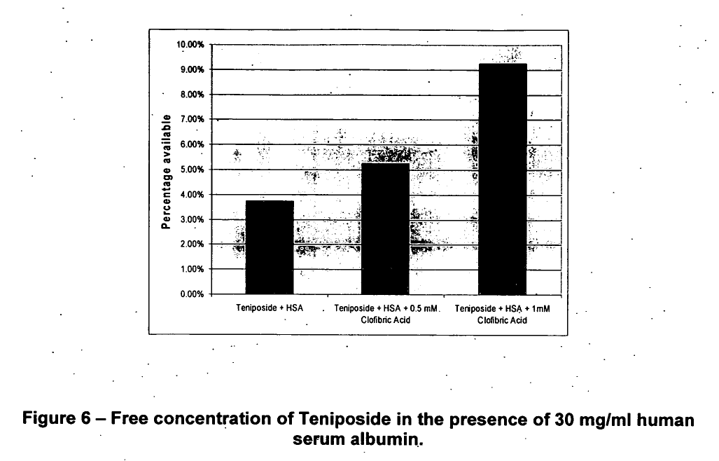

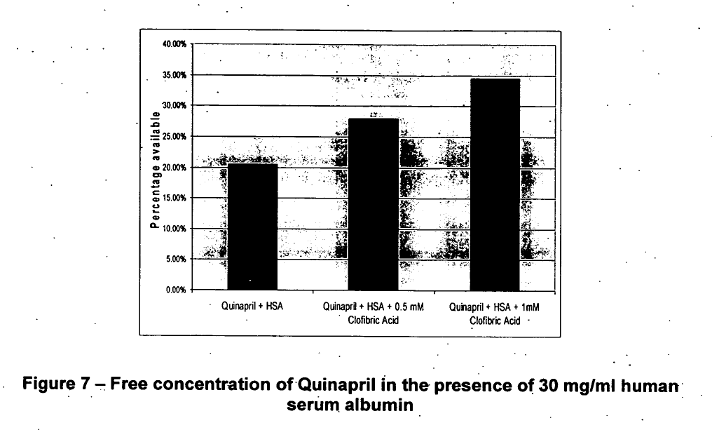

To validate this approach can also improve the circulatory availability of other therapeutic drugs, we have carried out free concentration measurement for two other drugs that bind in IB pocket of human serum albumin, Teniposide, an anti-cancer drug (FIG. 6), Quinapril, an anti-hypertensive drug (FIG. 7), and Sulfisoxazole, an antibiotic or anti-infective drug (FIG. 8). As we predicted, after three hours of incubation, the free concentration of all of these drugs are significantly higher in the presence of 0.5 mM and 1 mM Salus agent such as clofibrate comparing with that in the absence of a displacement agent.

The examples shown above not only once again validate the power of the Salus™ technology, but has tremendous implications in clinical cancer therapy. Camptothecin, being toxic only to the cells in S-phase, must be exposed to the cancer cells over a prolonged period of time at high level to be effective. Our findings clearly show that the co-administration of Salus™ agent(s) highly bound to the identified Camptothecin site, can achieve dramatically enhanced therapeutic profiles for this family of drugs. Since the preferred Salus™ agent represents a highly safe FDA approved pharmaceutical, clinical studies could be accelerated.

### APPENDIX 1

The following reflects the test protocol for the cytotoxicity studies:

Cytotoxicity of DRUG for Breast Cancer Cells (MDA-MB-435S)

Initial Setup:

1. Count number of cells in flask. The final concentration of cells in each well should be between 5000-40000 cells/well (100,000-800,000 cells/ml). Dilute cells to proper concentration using Leibovitz's L-15 supplemented with 10% FBS and 0.1% insulin. Add 100 μl of cells into the microplate using repeating pipette. Be sure to vortex tube before adding to plate. Allow plate to incubate for 24 hours at 37° C. (Note: wells A1-H1, A1-H11 will contain only medium for evaporation purposes. Well A2 will contain no cells, only medium and will be used in experiment.)
   Medium: Leibovitz's L-15 medium.
2. Albumin:
   Make 60 mg/ml in HSA (900 mg HSA+13.3 ml medium). Use Sigma HSA A-8763
   Make 5 ml of 60 mg/ml HSA with 2 mM 301 (2.33 mg 301 for 5.4 ml HSA).
   Make 1 ml of 2 mM 301 in medium (0.53 mg in 1.23 ml medium). Dilute 200 μl in 200 μl medium to make 1 mM 301.
   Make DRUG stock in DMSO Stock molarity=1000× conc. of highest test sample (ex. if 2 μM is needed, then stock needs to be 2 mM.) This will vary with each drug based on dosage of drug. For Cancer Cells, the highest concentration should be 1-2 times greater than the dosage for the drug.
3. Make the test concentrations in advance of testing (5-24 hours prior to testing): The test concentrations will be made at double the concentration, 100 μl will be added into the 100 μl of the cells, making the final concentrations the same as those shown on the microplate chart. Change medium in plate (using the L-15 supplemented with 10% FBS and 0.1% insulin prior to loading test concentrations.
   100% cells: 100 μl of medium
   medium: 100 μl of medium
   0.5 mM 301: add 100 μl of 1 mM 301 made in medium
   0.5 mM 301 with HSA: add 100 μl of 1 mM 301 with 30 mg/ml HSA
   1 mM 301: add 100 μl of 2 mM 301 made in medium
   1 mM 301 with HSA: add 100 μl of 2 mM 301 with 30 mg/ml HSA
   30 mg/ml HSA: add 100 μl of 60 mg/ml HSA made in medium (2 wells)
   Dosage of DRUG: make a 1:500 dilution, using medium as the diluent. Make 2-fold serial dilutions into 300 μl of medium (600 μl total volume). Be sure to make dilutions before adding into wells.
   30×HSA & Dosage DRUG: Follow same serial dilution procedure as above with the DRUG, but the diluent should be medium +60 mg/ml HSA
   30×HSA, 0.5 mM 301 & DRUG: Follow same serial dilution procedure as above with the DRUG, but the diluent should be medium +60 mg/ml HSA with 1 mM 301
   30×HSA, 1 mM 301 & 2 μM DRUG: Follow same serial dilution procedure as above with the DRUG, but the diluent should be medium +60 mg/ml HSA with 2 mM 301.
Allow these to incubate at 37° C. overnight. After the microplate has been incubating for 24 hours, add the above test concentrations into the appropriate well (see Chart for microplate information). Allow plate to incubate an additional 48 hours.
   After 48 Hours:
4. Under sterile hood, reconstitute the XTT vial with 5 ml of medium.
5. Remove all liquid from wells. Wash with medium, remove, then fill wells with 200 μl of fresh medium.
6. Add 40 μl of XTT to all wells.
7. Allow wells to incubate 2 hours.
8. Read the plate in the microplate reader using a 655 nm reading as a reference wavelength. The read at 450 nm. Subtract the 655 nm reading from the 450 reading.

The tray set up for the cytotoxicity studies is included below.

| | 1 | 2 | 3 | 4 | 5 | 6 | 7 |
|---|---|---|---|---|---|---|---|
| A | medium | medium | conc of | conc of | 30X HSA & | 30X HSA & | 30X HSA, |
| | | | DRUG | DRUG | conc of DRUG | conc of DRUG | 0.5 mM 301& |
| | | | | | | | conc of DRUG |
| B | medium | 100% cells | half of conc | half of conc | 30X HSA & half | 30X HSA & half | 30X HSA, |
| | | | in A3 of | in A4 of | of conc in A5 of | of conc in A6 of | 0.5 mM 301& |
| | | | DRUG | DRUG | DRUG | DRUG | half of conc in |
| | | | | | | | A7 of DRUG |
| C | medium | 0.5 mM 301 | half of conc | half of conc | 30X HSA & half | 30X HSA & half | 30X HSA, |
| | | | in B3 of | in B4 of | of conc in B5 of | of conc in B6 of | 0.5 mM 301& |
| | | | DRUG | DRUG | DRUG | DRUG | half of conc in |
| | | | | | | | B7 of DRUG |
| D | medium | 1 mM 301 | half of conc | half of conc | 30X HSA & half | 30X HSA & half | 30X HSA, |
| | | | in C3 of | in C4 of | of conc in C5 of | of conc in C6 of | 0.5 mM 301& |
| | | | DRUG | DRUG | DRUG | DRUG | half of conc in |
| | | | | | | | C7 of DRUG |
| E | medium | 0.5 mM 301 | half of conc | half of conc | 30X HSA & half | 30X HSA & half | 30X HSA, |
| | | & 30X HSA | in D3 of | in D4 of | of conc in D5 of | of conc in D6 of | 0.5 mM 301& |
| | | | DRUG | DRUG | DRUG | DRUG | half of conc in |
| | | | | | | | D7 of DRUG |
| F | medium | 1 mM 301 & | half of conc | half of conc | 30X HSA & half | 30X HSA & half | 30X HSA, |
| | | 30X HSA | in E3 of | in E4 of | of conc in E5 of | of conc in E6 of | 0.5 mM 301& |
| | | | DRUG | DRUG | DRUG | DRUG | half of conc in |
| | | | | | | | E7 of DRUG |
| G | medium | 30X HSA | half of conc | half of conc | 30X HSA & half | 30X HSA & half | 30X HSA, |
| | | | in F3 of | in F4 of | of conc in F5 of | of conc in F6 of | 0.5 mM 301& |
| | | | DRUG | DRUG | DRUG | DRUG | half of conc in |
| | | | | | | | F7 of DRUG |
| H | medium | 100% cells | half of conc | half of conc | 30X HSA & half | 30X HSA & half | 30X HSA, |
| | | | in G3 of | in G4 of | of conc in G5 of | of conc in G6 of | 0.5 mM 301& |
| | | | DRUG | DRUG | DRUG | DRUG | half of conc in |
| | | | | | | | G7 of DRUG |

| | 8 | 9 | 10 | 11 | 12 |
|---|---|---|---|---|---|
| A | 30X HSA, | 30X HSA, 1 mM | 30X HSA, 1 mM | medium | Blank |
| | 0.5 mM 301& | 301& conc of DRUG | 301& conc of | | |
| | conc of DRUG | | DRUG | | |
| B | 30X HSA, | 30X HSA, 1 mM | 30X HSA, 1 mM | medium | Blank |
| | 0.5 mM 301& | 301& half of conc in | 301& half of | | |
| | half of conc in | A9 | conc in A10 | | |
| | A8 of DRUG | | | | |
| C | 30X HSA, | 30X HSA, 1 mM | 30X HSA, 1 mM | medium | Blank |
| | 0.5 mM 301& | 301& half of conc in | 301& half of | | |
| | half of conc in | B9 | conc in B10 | | |
| | B8 of DRUG | | | | |
| D | 30X HSA, | 30X HSA, 1 mM | 30X HSA, 1 mM | medium | Blank |
| | 0.5 mM 301& | 301& half of conc in | 301& half of | | |
| | half of conc in | C9 | conc in C10 | | |
| | C8 of DRUG | | | | |
| E | 30X HSA, | 30X HSA, 1 mM | 30X HSA, 1 mM | medium | Blank |
| | 0.5 mM 301& | 301& half of conc in | 301& half of | | |
| | half of conc in | D9 | conc in D10 | | |
| | D8 of DRUG | | | | |
| F | 30X HSA, | 30X HSA, 1 mM | 30X HSA, 1 mM | medium | Blank |
| | 0.5 mM 301& | 301& half of conc in | 301& half of | | |
| | half of conc in | E9 | conc in E10 | | |
| | E8 of DRUG | | | | |
| G | 30X HSA, | 30X HSA, 1 mM | 30X HSA, 1 mM | medium | Blank |
| | 0.5 mM 301& | 301& half of conc in | 301& half of | | |
| | half of conc in | F9 | conc in F10 | | |
| | F8 of DRUG | | | | |
| H | 30X HSA, | 30X HSA, 1 mM | 30X HSA, 1 mM | medium | Blank |
| | 0.5 mM 301& | 301& half of conc in | 301& half of | | |
| | half of conc in | G9 | conc in G10 | | |
| | G8 of DRUG | | | | |

TABLE I
Compounds determined crystallographically
which include Site IB Specificity

| Indication | Drug |
|---|---|
| Anti-(prostate) cancer, NSAID | Bicalutamide |
| Antiamyotrophic lateral sclerosis, | Riluzole |
| anticonvulsant | |
| Anti-arrhythmic and/or anesthetic | Lidocaine |
| | Quinidine Gluconate |
| Antibiotic | Ampicillin |
| | Metampicillin |
| | Sulfisoxazole |
| Anti-cancer | 9-aminoCamptothecin |
| | Camptothecin |
| | Idarubicin |
| | Teniposide |
| Anti-coagulant | Dicumarol |
| Anti-convulsant | Methsuximide |
| Anti-depressant | Trazodone |
| Anti-diabetic | Tolbutamide |
| | Glimepiride |
| | Glipizide |
| | Glyburide |
| Anti-histamine | Fexofenadine |
| Anti-hypertensive and/or ACE inhibitor | Alprenolol |
| | Chlorothiazide |
| | Doxazosin |
| | Hydralazine |
| | Irbesartan |
| | Methyldopate |
| | Prazosin |
| | Quinapril |
| | Ramipril |
| | Telmisartan |
| | Terazosin |
| | Valsartan |
| Anti-infective | Cefamandole Nafate |
| | Nalidixic Acid |
| Anti-inflammatory | Budesonide |
| | Ketorolac |
| Anti-lipemic | Fenofibric Acid |
| Anti-porphyria | Hemin |
| Anti-psychotic | Ziprasidone |
| Cholesterol Lowering | Cerivastatin |
| | Clofibric Acid |
| | Gemfibrozil |
| Contraceptive steroid | Norethindrone |
| Dye | Resazurin |
| Estrogen Replacement | Ethinyl Estadiol |
| Hepatic function diagnostic aid | Sulfobromophthalein |
| NSAID and/or Analgesic | Diflunisal |
| | Etodolac |
| | Fenoprofen |
| | Ibuprofen |
| | Ketoprofen |
| | N-(2-Nitrophenyl)-anthranilic |
| | Acid |
| | Nambutone |
| | Naproxen |
| | N-Phenylanthranilic Acid |
| | Tolmetin |
| Skeletal muscle relaxant | Chlorzoxazone |
| | Cyclobenzaprine |
| Stimulant | Caffeine |
| Others | Arachidonic Acid |
| | Linoleic Acid |
| | Palmitic Acid |
| | Palmitoleic Acid |
| | Stearic Acid |

TABLE II
Salus IB Agents In Accordance
With the Invention

| Indication | Drug | Dosage | MW |
|---|---|---|---|
| NSAID and/or Analgesic | Diflunisal | (500 mg-1000 mg/day) | 250.2 |
| | Etodolac | (1000 mg/day) | 287.4 |
| | Fenprofen | (900 mg-2400 mg/day) | 522 |
| | Ibuprofen | (1200 mg-3200 mg/day) | 228.3 |
| | N-(2-Nitrophenyl)-anthranalic Acid | | |
| | Nambutone^{§} | (2000 mg/day) | 216.2 |
| | Naproxen | (1000 mg/day) | 252.25 |
| | N-Phenylanthranalic Acid | | 213.24 |
| | Tolmetin | (600 mg-1800 mg/day) | 315.3 |
| Cholesterol Lowering | Clofibric Acid* | (2000 mg/day) | 214.65 |
| | Gemfibrozil | (1200 mg/day) | 250.3 |
| Anti-infective | Ampicillin | (2000 mg-3500 mg/day) | 371.4 |
| | Cefamandole Nafate | (12 g/day max) | 512.5 |
| | Nalidixic Acid | (4000 mg/day) | 232.2 |
| Anti-hypertensive and/or Diuretic | Chlorothiazide | (1000 mg-2000 mg/day) | 295.7 |
| | Methyldonate | (500 mg-2000 mg/day) | 275.73 |
| Anti-arrhythmic and/or Anti-malarial | Quinidine Gluconate | (648 mg/8 hrs) | 520.6 |

^{§}Nambutone or 6-methoxy-2-naphthylacetic acid (6MNA), form depending on route of administration (active form 6MNA)
*Clofibrate or Clofibric acid, form depending on route of administration (active form clofibric acid)

What is claimed is:

1. A method for maximizing the therapeutic effectiveness of a drug that reduces the level of rapidly dividing cells in a patient in need thereof and that binds at the IB site of human serum albumin comprising co-administering a compound that binds competitively with said drug at the IB site of human serum albumin in an amount effective to maximize the therapeutic effectiveness of said drug in the patient.

2. The method according to claim 1, wherein the competitive compound binding at the IB site of human serum albumin is selected from the group consisting of clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate.

3. The method according to claim 1, wherein the competitive compound achieves a plasma concentration in patient in the range of about 0.1 mM to 25.0 mM.

4. The method according to claim 1, wherein the competitive compound binding at the IB site of human serum albumin is administered intravenously, by intraperitoneal or subcutaneous injection, or orally.

5. The method according to claim 1, wherein the competitive compound binding at the IB site of human serum albumin is administered before, simultaneously with, or after administration of the albumin-binding drug.

6. The method according to claim 1, wherein the competitive compound has a higher affinity to human serum albumin at the IB site of human serum albumin than the albumin-binding drug in the bloodstream.

7. The method according to claim 1, wherein the competitive compound can block the albumin-binding drug from binding at the IB site of human serum albumin in the bloodstream.

8. The method according to claim 1, wherein the competitive compound can displace the albumin-binding drug at the IB site of human serum albumin in the bloodstream.

9. The method according to claim 1, wherein the IB-binding drug is selected from the group consisting of the Camptothecin family of drugs, including but not limited to Camptothecin, 10-hydroxycamptothecin, 9-aminocamptothecin, Topotecan and Irrinotecan, the anthracyclin family of drugs including but not limited to Doxorubicin and Epirubicin, the Taxol family of drugs including but not limited to Paclitaxol, the Etoposide family of drugs and the Teniposide family of drugs.

10. The method according to claim 1, wherein the competitive compound is an agent that can reduce the level of rapidly dividing cells in a patient.

11. The method according to claim 1, wherein the administration of the competitive compound maximizes the therapeutic index of the drug that reduces the level of rapidly dividing cells in a patient.

12. A method for increasing the free concentration of a drug that reduces the level of rapidly dividing cells in a patient in need thereof and that binds at the IB site of human serum albumin comprising administering the drug in the presence of a compound that binds competitively with said drug at the IB site of human serum albumin in an amount effective to increase the free concentration of said drug in the bloodstream of the patient.

13. The method according to claim 12, wherein the competitive compound binding at the IB site of human serum albumin is selected from the group consisting of clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate.

14. The method according to claim 12, wherein the competitive compound binding at the IB site of human serum albumin is administered intravenously, by intraperitoneal injection, subcutaneous injection, or orally.

15. The method according to claim 12, wherein the competitive compound is administered before, simultaneously with, or after administration of the IB-binding drug.

16. The method according to claim 12, wherein the IB-binding drug is selected from the group consisting of the Camptothecin family of drugs, including but not limited to Camptothecin, 10-hydroxycamptothecin, 9-aminocamptothecin, Topotecan and Irrinotecan, the anthracyclin family of drugs including but not limited to Doxorubicin and Epirubicin, the Taxol family of drugs including but not limited to Paclitaxol, the Etoposide family of drugs and the Teniposide family of drugs.

17. The method according to claim 12, wherein the competitive drug reduces the affinity of the albumin-binding drug to other binding sites on human serum albumin.

18. A therapeutic composition for reducing the level of rapidly cells in a patient in need thereof, comprising a drug that reduces the level of rapidly dividing cells in a patient and that binds to human serum albumin at the IB site and a compound that competitively binds to human serum albumin in an amount effective to increase or mudulate the free concentration of the drug in the bloodstream of a patient in need.

19. The composition of claim 18, wherein the competitive compound binding at the IB site of human serum albumin is selected from the group consisting of clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate.

20. The composition of claim 18, wherein the IB-binding drug is selected from the group consisting of the Camptothecin family of drugs, including but not limited to Camptothecin, 10-hydroxycamptothecin, 9-aminocamptothecin, Topotecan and Irrinotecan, the anthracyclin family of drugs including but not limited to Doxorubicin and Epirubicin, the Taxol family of drugs including but not limited to Paclitaxol, the Etoposide family of drugs and the Teniposide family of drugs.

21. The composition of claim 18, further comprising a pharmaceutically acceptable vehicle, carrier or excipient.

22. A method for maximizing the therapeutic effectiveness of a drug that reduces hypertension comprising administering an anti-hypertensive drug that binds to human serum albumin at the IB site along with a compound that binds competitively with said drug at the IB site of human serum albumin in an amount effective to manage the reduction of hypertension in the patient.

23. The method according to claim 22, wherein the competitive compound binding competitively at the IB site of human serum albumin is selected from the group consisting of clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate.

24. The method according to claim 22, wherein the drug is selected from the group consisting of Prazosin, Ramapril, Quinapril, Terazosin, Hydralazine, Methyldopate. Valsartan, Irbesartan, Alprenolol, Chlorothiazide and Doxazosin.

25. The method according to claim 22, wherein the competitive compound increases the free concentration of the anti-hypertensive drug in the patient

26. A method of maximizing the therapeutic effectiveness of an anti-infective drug that binds to the IB site of human serum albumin comprising administering with said drug a compound that binds competitively with said drug at the IB site of human serum albumin in an amount effective to maximize the effectiveness of the anti-infective drug.

27. The method according to claim 26, wherein the compound binding competitively at the IB site of human serum albumin is selected from the group consisting of clofibrate, clofibric acid, Tolmetin, Fenoprofen, Diflunisal, Etodolac, Naproxen, Nambutone, Ibuprofen, Chlorothiazide, Gemfibrozil, Nalidixic Acid, Methyldopate, Ampicillin, Cefamandole Nafate, N-(2-Nitrophenyl)-anthranilic Acid, N-Phenylanthranilic Acid and Quinidine Gluconate.

28. The method according to claim 26 wherein the anti-infective drug is selected from the group consisting of Ampicillin, Metampicillin, Sulfisoxazole, Nalidixic Acid and Cefamandole Nafate.
